(19) United States
(12) Patent Application Publication
(10) Pub. No.: US 2020/0341005 A1
(43) Pub. Date: Oct. 29, 2020

(54) Carrier and method for detecting an analyte in dried blood spots

(71) Applicant: EUROIMMUN Medizinische Labordiagnostika AG, Luebeck (DE)

(72) Inventors: Henning SEISMANN, Nienwohld (DE); Victor Herbst, Krummesse (DE); Alf Weimann, Luebeck (DE); Birgit Wichner, Luebeck (DE); Winfried Stoecker, Gross Groenau (DE)

(73) Assignee: EUROIMMUN Medizinische Labordiagnostika AG, Luebeck (DE)

(21) Appl. No.: 15/929,283

(22) Filed: Apr. 22, 2020

(30) Foreign Application Priority Data
Apr. 25, 2019 (EP) 19171226.4

(51) Int. Cl. G01N 33/68; G01N 33/49

(52) U.S. Cl. CPC G01N 33/6854; C08L 23/12; G01N 2800/24; G01N 33/49

(57) ABSTRACT

A carrier includes at least one take-up region based on a non-woven polyolefin containing dried-up capillary blood. The carrier is useful for semi-quantitatively, preferably quantitatively determining the concentration of an analyte in a blood sample. Further, a method is used for detecting an analyte in a blood sample and includes the steps of providing the carrier, applying a blood sample to the take-up region of the carrier, drying the carrier, contacting the carrier containing the dried-up capillary blood with a liquid which is suitable for taking up the analyte from the dried-up blood sample, and detecting the analyte in the liquid.

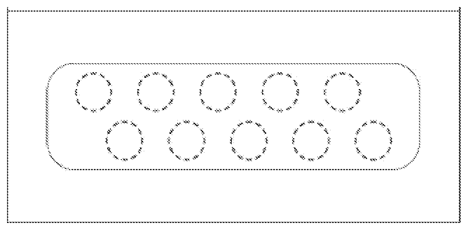

### CROSS-REFERENCE TO RELATED APPLICATION

The present application claims the benefit to European application EP 19 171226.4, filed on Apr. 25, 2019, the content of which is hereby incorporated by reference in its entirety.

### BACKGROUND OF THE INVENTION

### Field of the Invention

The present invention relates to a carrier comprising at least one take-up region based on a non-woven polyolefin containing dried-up capillary blood, to the use of the carrier for semi-quantitatively, preferably quantitatively determining the concentration of an analyte in a blood sample, and to a method for detecting an analyte in a blood sample comprising the steps of providing the carrier, applying a blood sample to the take-up region of the carrier, drying the carrier, contacting the carrier containing the dried-up capillary blood with a liquid which is suitable for taking up the analyte from the dried-up blood sample, and detecting the analyte in the liquid.

### Discussion of the Background

For many qualitative determinations in the field of analytical chemistry and specifically laboratory diagnostics, what only matters is that the presence or absence of an analyte can be correctly established, but not the concentration in which it is present in the blood of a patient. For example, this is the case when the blood sample is tested to establish whether the patient is suffering from a metabolic disorder associated with a defective gene. The blood sample must then be merely tested to establish whether such a gene is present, but not how much of the genetic material comprising the gene is contained in the sample.

In the case of other analytes, what has to be established is the concentration in which the analyte occurs in the blood, because a diagnostic reference concentration range is known. If the concentration of the analyte is within said range, this indicates that the patient is healthy. For such quantitative determinations, what therefore matters is that the amount or concentration of the analyte is correctly determined.

In the case of a quantitative determination, what are to be avoided are all sample material processing steps that lead to an alteration of the sample material that causes a change to the signal level when measuring the analyte, which signal level indicates the concentration of the analyte.

There are numerous possibilities as to how the processing of a sample can alter the signal. Apart from a possible loss of the analyte during processing, it is also quite possible that what occurs at the same time is an alteration of, for example, the chemical nature of the analyte, which leads to the same amount of analyte yielding a different signal, this indicating an excessively high or excessively low amount of the analyte. A further possibility is that, during processing, an external chemical substance is added to the sample material or formed, which external chemical substance generates a similar signal during measurement to the analyte of interest and thus increases the overall signal or which external chemical substance suppresses the signal of the analyte. Lastly, is it possible that varying processing conditions cause a fluctuation in the measured signal.

For the measurement of concentrations of certain analytes in the blood of patients, venous blood is usually drawn from the patient, followed by the treatment of the blood to yield serum. Apart from the fact that this invasive procedure is unpleasant for the patient and even involves health risks to a small extent, such as the risk of nausea or fainting, the drawing of blood can only be carried out by a qualified specialist such as a physician or at least a nurse or experienced laboratory technician. To this end, the patient has to visit a medical practice or a hospital.

By contrast, obtaining capillary blood is much simpler and gentler. After the fingertip or the earlobe of the patient has been pricked with a sharp object, a few drops of capillary blood escape, which are taken up with a pipette and transferred to an absorptive carrier. Once the blood has dried up on the carrier, the carrier can be transported very easily, even by mail, without further treatment. The visit to the medical practice thus becomes unnecessary. From a carrier containing dried-up blood, preferably capillary blood, it is possible to detach a piece of carrier containing blood. For example, this can be achieved using a device as described in U.S. Ser. No. 14/900,360 or by punch-out. The detached piece of carrier containing dried-up blood is commonly referred to as a “dried blood spot” or “DBS”.

Whereas this way of obtaining samples especially for qualitative methods has already been established for years, a lack of sharpness still presents itself in quantitative determinations of some analytes. In particular, background signals, which make an accurate quantification more difficult, occur when determining antibodies from capillary blood using conventional carriers. This affects the usability of dried blood spots.

A problem addressed by the present invention is therefore that of providing carriers and methods which allow a blood sample, preferably capillary blood sample, to be processed and be used for determination in such a way that the signal, the level of which indicates the concentration of the analyte of interest, corresponds at least sufficiently in absolute or relative terms with the signal obtained from the same patient when the analyte is determined from blood serum.

A further problem addressed by the present invention is that of increasing the reproducibility of quantitative determinations of analytes from capillary blood by providing suitable carriers and methods. Using capillary blood and the carrier and method according to the invention, the aim is to obtain determination values which are representative of the concentration of the analyte in the blood as would have been obtained using venous blood for determination.

A further problem addressed by the present invention is that of providing carriers and methods for quantitatively determining analytes from capillary blood, in which the measurement of the signal indicating the concentration of the analyte is made possible with background signal interference that is reduced compared to the related art or is tolerable. What is to be provided is a carrier usable for this purpose.

U.S. Pat. No. 9,110,053 discloses a range of materials for dried blood spots, namely cellulose-based materials, glass fibres, composite materials composed of glass fibres and cellulose, nylon, polyester, polypropylene, nitrocellulose, modified polyether, polyvinyl chloride, natural or synthetic fibres or laminated materials. It is pointed out that cellulose-free glass fibres increase the capacity for determining small molecules, especially for pharmaceutical analysis.

Kumar et al. disclose the detection of IgG for serodiagnosis of human hydatidosis using dried blood spots on filter paper (Kumar. N., Sehgal. R., Goyal, K., and Tripathi, P. (2012) Evaluation of dried blood spots collected on filter paper for serodiagnosis of human hydatidosis by enzyme-linked immunosorbent assay, Trop. Parasitol. 2(2), 119-123, 2012).

Jamaly et al. disclose an assay in which blood serum containing the CAA antigen of Schistosoma is contacted and the antigen taken up is subsequently quantified using an ELISA based on a CAA-specific monoclonal antibody (Jamaly. S. et al., Transactions of the Royal Society of Tropical Medicine and Hygiene (1997) 91, 412-415). In a comparison of various absorption materials, especially non-woven polypropylene fibres, with various filter papers, Jamaly et al. reached the conclusion that the highest yield is achieved using the polypropylene fibres. The reproducibility in comparison with an ELISA in which serum is used as sample material was not tested.

### SUMMARY OF THE INVENTION

The problem addressed by the invention is solved by the subject matter of the following embodiments.
1. Carrier comprising at least one take-up region based on a non-woven polyolefin containing dried-up capillary blood.
2. Drawing set comprising a carrier comprising at least one take-up region based on a non-woven polyolefin, a sterile pricking aid for drawing capillary blood and optionally a pipette.
3. Use of a carrier comprising at least one take-up region based on a non-woven polyolefin or use of the take-up region for semi-quantitatively, preferably quantitatively determining the concentration of an analyte in a blood sample.
4. Method for detecting an analyte in a blood sample, comprising the steps of
   a) providing a carrier comprising at least one take-up region based on a non-woven polyolefin,
   b) applying a blood sample to the take-up region,
   c) drying the carrier,
   d) contacting the carrier or the take-up region thereof comprising the dried-up blood sample from step c) with a liquid which is suitable for taking up the analyte from the dried-up blood sample, and
   e) detecting the analyte in the liquid.
5. Carrier, drawing set, use or method according to any of embodiments 1 to 4, wherein the at least one take-up region is surrounded by a material having lower absorptivity than that of the take-up region, preferably cardboard.
6. Carrier, drawing set, use or method according to any of embodiments 1 to 5, wherein the carrier comprises at least two take-up regions based on a non-woven polyolefin that are separated from one another, preferably by a region based on a material having lower absorptivity than that of the take-up region, preferably cardboard.
7. Use or method according to any of embodiments 3 to 6, wherein the blood sample is capillary blood.
8. Carrier, drawing set, use or method according to any of embodiments 1 to 7, wherein the capillary blood is untreated capillary blood.
9. Carrier, drawing set, use or method according to any of embodiments 1 to 8, wherein the polyolefin is polypropylene.
10. Use or method according to any of embodiments 3 to 9, wherein the detection is a semi-quantitative detection, preferably a quantitative detection.
11. Use or method according to any of embodiments 3 to 10, serving for the diagnosis of a food allergy or food intolerance.
12. Use or method according to any of embodiments 3 to 11, wherein the detection or the determination is carried out using a diagnostically useful carrier comprising a means for specifically capturing an antibody.
13. Use or method according to embodiment 12, wherein the diagnostically useful carrier is selected from the group comprising a blot; preferably line blot, dot blot or western blot; a bead, a microtiter plate, a test strip, a microarray, a glass slide, a biochip and a lateral flow device.
14. Use or method according to any of embodiments 3 bis 13, wherein the analyte is an antibody, preferably an antibody of the IgG class.
15. Use or method according to embodiment 14, wherein the antibody is an antibody against an allergen-containing and/or protein-containing material based on a foodstuff, preferably from the group comprising courgette, pepper, chicken meat, codfish, salmon, walnut, watermelon, kiwi fruit, guinea fowl, rabbit, crab, rose fish, chives and papaya.

### BRIEF DESCRIPTION OF DRAWING

The FIGURE shows schematically a carrier consisting of one of the above-described membranes containing marked sample-application regions, which membrane is applied to a cardboard carrier. The whole blood is dripped into the region of the marked circles.

### DETAILED DESCRIPTION OF THE INVENTION

In a first aspect, the problem is solved by a carrier, preferably for detecting an analyte in a blood sample, comprising at least one take-up region based on a non-woven polyolefin containing dried-up capillary blood.

In a second aspect, the problem is solved by a drawing set comprising a carrier, preferably for detecting an analyte in a blood sample, comprising at least one take-up region based on a non-woven polyolefin, a sterile pricking aid for drawing capillary blood and optionally a pipette.

In a third aspect, the problem is solved by the use of a carrier, preferably for detecting an analyte in a blood sample, comprising at least one take-up region based on a non-woven polyolefin or use of the take-up region for semi-quantitatively, preferably quantitatively determining the concentration of an analyte in a blood sample.

In a fourth aspect, the problem is solved by a method for detecting an analyte in a blood sample, comprising the steps of
a) providing a carrier comprising at least one take-up region based on a non-woven polyolefin,
b) applying a blood sample to the take-up region,
c) drying the carrier,
d) contacting the carrier or the take-up region thereof comprising the dried-up blood sample from step c) with a liquid which is suitable for taking up the analyte from the dried-up blood sample, and
e) detecting the analyte in the liquid,
wherein the analyte is preferably an antibody and the detection or the determination of the analyte is carried out using a diagnostically useful carrier comprising a means for specifically capturing the antibody.

In a preferred embodiment, the at least one take-up region is surrounded by a material having lower absorptivity than that of the take-up region, the material being preferably cardboard.

In a preferred embodiment, the carrier comprises at least two take-up regions based on a non-woven polyolefin that are separated from one another, preferably by a region based on a material having lower absorptivity than that of the take-up regions, the material being preferably cardboard.

In a preferred embodiment, what is concerned is a carrier comprising at least one take-up region based on a non-woven polyolefin containing dried-up capillary blood for providing a sample for the detection of an analyte contained therein.

In a further preferred embodiment, what is concerned is the use of a carrier comprising at least one take-up region based on a non-woven polyolefin, the at least one take-up region comprising dried-up capillary blood as taken-up blood sample, for providing a sample for the detection of an analyte contained therein.

In a preferred embodiment, the blood sample is capillary blood.

In a preferred embodiment, the capillary blood is untreated capillary blood.

In a preferred embodiment, the polyolefin is polypropylene.

In a preferred embodiment, the detection is a semi-quantitative detection, preferably a quantitative detection. In a further preferred embodiment, the detection is a qualitative detection.

In a preferred embodiment, the use or the method serves for the diagnosis of a food allergy or food intolerance.

In a preferred embodiment, the detection or the determination is carried out using a diagnostically useful carrier comprising a means for specifically capturing an antibody.

In a preferred embodiment, the diagnostically useful carrier is selected from the group comprising a blot; preferably line blot, dot blot or western blot; a bead, a microtiter plate, a test strip, a microarray, a glass slide, a biochip and a lateral flow device.

In a preferred embodiment, the analyte is an antibody, preferably an antibody of the IgG class.

In a preferred embodiment, the antibody is an antibody against an allergen-containing and/or protein-containing material based on a foodstuff, preferably from the group comprising courgette, pepper, chicken meat, codfish, salmon, walnut, watermelon, kiwi fruit, guinea fowl, rabbit, crab, rose fish, chives and papaya.

The present invention is based on the surprising finding by the inventors that non-woven polyolefin fibres as means for taking up an aqueous sample, which then dries up thereon, have, after the material has been taken up into another aqueous medium, the advantageous property that analytes from the aqueous sample can be quantitatively determined with a result which particularly exactly corresponds with that directly determined from a serum sample. This concerns especially the quantitative determination of antibodies as analyte.

In a preferred embodiment, the term “analyte”, as used herein, is understood to mean a substance which is present in the blood sample and which remains on the take-up region of the carrier when the blood sample has dried up and can be transferred from there into another liquid for quantitative determination. Particular preference is given to substances which are readily soluble in aqueous solutions and which were dissolved in the blood sample. However, also possible are substances such as solid particles which are present in solution in the form of a suspension. Their concentration, too, in the blood sample or an aqueous solution can be quantitatively determined using suitable physical measurement methods such as the detection of light scattering. In a particularly preferred embodiment, the analyte is selected from the group comprising a metabolite, a protein, a nucleic acid and a lipid and is particularly preferably an antibody, yet more preferably selected from the group of classes comprising IgA, IgM, IgG and IgE, most preferably IgG. In a preferred embodiment, the antibody is an antibody from a mammal, yet more preferably from a human.

In a preferred embodiment, the sample is a sample from a mammal, yet more preferably from a human.

The carrier is a solid carrier which comprises a take-up region. The take-up region comprises a material which absorbs an aqueous blood sample and is based on polyolefin, and preferably comprises non-woven polyolefin having a mass fraction of at least 5%, 10%, 20%, 30%, 40%, 50%, 60%, 70%, 80%, 90% or 95%. The take-up region is constituted and separated from other absorptive parts of the carrier such that a spatial spreading of the dripped-on blood sample across the carrier is confined. It is preferably directly geometrically accessible for drip-on of the blood sample. In the event that the carrier comprises two, three, four, six, eight, 10, 12, 20, 24, 48 or more take-up regions, these are preferably separated from one another such that one blood sample dripped into one take-up region cannot contaminate other take-up regions, particularly in the event that multiple blood samples from more than one patient are dripped on. A take-up region is preferably laterally surrounded by a hydrophobic material and/or a material having hydrophobic properties, and yet more preferably also on its lower side, i.e. preferably the side which is not the side from which the blood sample is added. The carrier can comprise a writeable or printable field for entering patient data and the desired analysis of the sample. It can furthermore comprise a machine-readable code, for example a barcode. The take-up region is preferably constituted such that it, together with a considerable portion of the dried-up blood sample, can be easily detached from the rest of the carrier, for example by separation from the rest in the form of a weakened line such as a perforation or by being connected to the rest of the carrier via a tear-off web.

The polyolefin is preferably selected from the group comprising polyethylene, polypropylene, polymethylpentene, polyisobutylene and polybutylene and is preferably polypropylene.

Besides the carrier, the drawing set preferably comprises a pricking aid for drawing capillary blood, which pricking aid is preferably sterile, and a pipette which is preferably a pipette having the dimensions for being able to introduce a few drops of blood and to deliver them onto the carrier.

In a preferred embodiment, the term “quantitative determination” is understood to mean a determination which affords information about the absolute concentration of the analyte, yet more preferably with a numerical value. The determination can be a semi-quantitative determination, which allows an assignment of the concentration to one range from at least three, preferably four concentration ranges, for example negative, weakly positive and positive, or a relative determination of concentration. In particular, the determination of concentration is carried out with the aid of calibrators, this preferably being two or more, preferably four units, preferably solutions or solid analytes coated on a diagnostically useful carrier, which each contain a known amount of the analyte, the two or more units comprising a different known amount in each case.

In a preferred embodiment, the term “absorptive”, as used herein, is understood to mean that the thus designated material is capable of taking up a drop of blood, wherein the water fraction is first taken up and then released to the environment upon drying, wherein the components dissolved therein, including the analyte, such as an antibody, remain in the material and can be dissolved out by repeated contacting with a suitable solvent, preferably an aqueous buffer. In a preferred embodiment, the take-up region consists of non-woven polyolefin, apart from the possible presence of commercial impurities or additives.

Preferably, the quantitative determination is carried out using a method selected from the group comprising immunodiffusion, immunoelectrophoresis, light scattering, agglutination and immunoassay with labelling—such as that from the group comprising immunoassay with radioactive labelling, with enzymatic labelling, more preferably ELISA, with chemiluminescence labelling, more preferably electrochemiluminescence labelling, and with immunofluorescence labelling, more preferably indirect immunofluorescence labelling—preferably with ELISA.

The method according to the invention concerns providing the carrier according to the invention, followed by applying a blood sample, followed by drying the carrier, which can be done by incubation at room temperature for a few minutes, preferably several hours or overnight. Alternatively, a carrier already comprising a dried-up blood sample is provided. According to the invention, the take-up region or a portion thereof containing at least some of the dried-up blood sample can be used for the further steps. To this end, this can be detached from the carrier, preferably by application of pressure across a perforated line, by punch-out or by tear-off. Devices are also known, by means of which a carrier containing multiple, non-detached take-up regions is subsequently contacted with multiple portions of a liquid or multiple liquids such that a cross-contamination of the various samples can be avoided, as described in US2012/0125127 for example. In a particularly preferred embodiment, the blood is blood from a patient.

In a particularly preferred embodiment, the drying step is carried out such that exclusively aqueous, volatile components of the sample, particularly preferably from the capillary blood, escape from the take-up region of the carrier, whereas non-volatile components, for example proteins, preferably antibodies, salts and similar compounds, remain on the carrier in the dry state. Especially water present in the sample is separated from the analytes dissolved therein and it is removed. Separation steps such as centrifugation, in which both liquid and non-liquid components are collected, but not separated from one another, are not a drying step. Optionally, the at least one take-up region can be separated from the carrier after drying. There is the possibility of subsequently dispatching it, for example by means of a courier or by post.

From the take-up region on the carrier on which the blood sample is dried up, said blood sample is eluted by contacting of the take-up region or the carrier with a liquid which is suitable for taking up the analyte from the dried-up blood sample. A possibility here is especially aqueous buffer having a suitable pH and salt content, for example PBS. The exact composition of the liquid and the conditions and the duration of the contacting can be found by routine stabilization and optimization experiments for the purpose of completest possible take-up of the analyte into the liquid and depend on the nature of the analyte. The liquid is preferably also equally chosen such that it is compatible with the subsequently used method for detecting the analyte.

Thereafter, the analyte of interest in the liquid is detected. This involves determining whether the analyte is present or absent or is present in a concentration above the detection limit of the detection method used. Preferably, the analyte is detected semi-quantitatively or quantitatively.

Various options for carrying out the method are described in the related art, for example Grüner, N., Stambouli, O. and Ross, R S. (2015) Dried Blood Spots—Preparing and Processing for Use in Immunoassays and in Molecular Techniques, J. Vis. Exp 97, 52619.

Preferably, one take-up region is separated from another take-up region via a region based on a material having a lower absorptivity than that of the take-up region. In a preferred embodiment, the term “absorptivity”, as used herein, means the ability to take up, relative to the mass, a highest possible volume of a liquid, preferably capillary blood or water, yet more preferably capillary blood. It can preferably be measured by adding, under the same conditions, identical volumes of the liquid to two equally heavy pieces of the two materials to be compared, until one of the materials no longer completely takes up the liquid.

The detection is preferably carried out using a diagnostically useful carrier comprising a means for specifically capturing the antibody. A bead as carrier can be a magnetic or fluorescent bead.

In a preferred embodiment, the term “means for specifically capturing an antibody”, as used herein, is understood to mean a reagent, antigen, peptide, protein, peptidomimetic or similar molecule which binds with sufficient binding strength to the antibody to be determined, it being more preferable that other antibodies are not bound to a considerable extent and remain in solution. It is yet more preferable when the binding is stronger than a binding reaction characterized by a dissociation constant which is 1×10⁻⁵ M, more preferably 1×10⁻⁷ M, more preferably 1×10⁻⁸ M, more preferably 1×10⁻⁹ M, more preferably 1×10⁻¹⁰ M, more preferably 1×10⁻¹¹ M, more preferably 1×10⁻¹² M, as determined by surface plasmon resonance using Biacore equipment at 25° C. in PBS buffer at pH 7. The antibody to be determined can be an antibody specifically against a certain antigen or a certain class of antibodies, preferably from the group comprising IgM, IgG, IgA or IgE, most preferably IgG. In the latter case, the means is preferably a means which is directed against the constant region of the antibody class to be captured. Appropriate means are known to a person skilled in the art and encompass secondary antibodies, protein A and protein G.

The subject matter of the present invention serves especially for the simple detection of IgG antibodies against food constituents, which detection is, however, at the same time more reliable compared to the related art, especially with respect to the correspondence between a semi-quantitative or quantitative determination of IgG from a sample containing capillary blood and a semi-quantitative or quantitative determination of IgG from a sample containing whole blood or serum, preferably serum. Thus, a more reliable indication of food intolerances and associated complaints is obtained using the subject matter of the present invention. It is not necessary for processed blood or blood provided with additives to be used. In particular, the blood sample does not have to contain heparin.

The invention is elucidated below on the basis of exemplary embodiments with reference to the figures. The embodiments described are merely exemplary in every respect and are not to be understood as limiting, and different combinations of the cited features are encompassed by the scope of the invention.

### Example

### Obtaining the Sample Material:

Whole blood was dripped onto different membranes (Ahlstrom 226—Ahlstrom-Munksjö, also referred to as “standard membrane”; Porex BNW441435—Porex Filtration Group, also referred to as “new membrane”) and dried at room temperature for at least four hours. The membranes were stored with desiccant in sealed-edge pouches at 4° C. and equilibrated to room temperature before each further use.

FIGURE shows schematically a carrier consisting of one of the above-described membranes containing marked sample-application regions, which membrane is applied to a cardboard carrier. The whole blood is dripped into the region of the marked circles.

### Extracting the Sample Material:

Punches sized 3/16 inch from the region of the circles marked in FIGURE, i.e. from the region containing the above-described membranes containing the applied and dried whole blood, were in each case provided with 1250 μl of universal buffer in a 1.5 ml screw-cap tube (Sarstedt AG; 1.5 ml microtube protein LB; article No. 72.694.600) and incubated at room temperature with shaking (orbital shaker; 400 rpm) for one hour and the liquid was then transferred to a new sample tube.

### Transferring the Extracted Sample Material to EUROLine IgG Food Test Strips:

For the incubation with EUROLine IgG Food Test Strips (Euroimmun Medizinische Labordiagnostika AG; DP 3022-0802-1 G; DP 3022-0802-2 G; DP 30244404-1 G; DP 3024-0404-2 G), 510 μl of the extracted sample material obtained in each case via one of the membranes were transferred to the test strips, which were equilibrated beforehand according to the information from the manufacturer. Further incubation was carried out according to the test instructions.

### Incubating the EUROLine IgG Food Test Strips and Comparing the Sample Materials:

To examine the comparability of the sample materials obtained via the two DBS methods with the standard sample material (serum), the EUROLine IgG Food Test Strips containing the two DBS sample materials (obtained via the “standard membrane” and obtained via the “new membrane”), obtained from the same patient in each case, were incubated according to the test instructions or as described above. The standard sample material (serum), from the same patient in each case, was likewise incubated according to the test instructions.

Validation was carried out using the EUROLineScan software and the results were compared on the basis of the ascertained class results (class 0-4), or on the basis of the ascertained band intensities.

### Result:

The raw data from this experiment are shown in Table 1, wherein all values which give a positive result with the standard membrane, but a negative result in the serum sample and the DBS sample with the new membrane, are marked.

TABLE 1
Raw data for the example

| | | | | Chicken | | |
|---|---|---|---|---|---|---|
| | | Courgette | Pepper | meat | Codfish | Salmon |
| Sample 1 | Serum | 2 | 8 | 57 | 4 | 6 |
| | Standard membrane | 6 | 10 | 49 | 8 | 13 |
| | New membrane | 5 | 8 | 50 | 5 | 7 |
| Sample 2 | Serum | 9 | 8 | 10 | 5 | 13 |
| | Standard membrane | 12 | 11 | 23 | 12 | 20 |
| | New membrane | 8 | 5 | 16 | 7 | 13 |
| Sample 3 | Serum | 38 | 4 | 9 | 4 | 14 |
| | Standard membrane | 31 | 9 | 18 | 8 | 18 |
| | New membrane | 32 | 5 | 12 | 6 | 15 |
| Sample 4 | Serum | 23 | 9 | 17 | 4 | 3 |
| | Standard membrane | 23 | 12 | 27 | 6 | 15 |
| | New membrane | 25 | 8 | 21 | 6 | 9 |
| Sample 5 | Serum | 4 | 4 | 6 | 7 | 6 |
| | Standard membrane | 12 | 10 | 19 | 8 | 14 |
| | New membrane | 7 | 6 | 8 | 8 | 9 |
| Sample 6 | Serum | 8 | 8 | 5 | 3 | 6 |
| | Standard membrane | 13 | 10 | 17 | 5 | 13 |
| | New membrane | 10 | 8 | 10 | 5 | 10 |
| Sample 7 | Serum | 23 | 31 | 12 | 4 | 5 |
| | Standard membrane | 21 | 26 | 24 | 8 | 13 |
| | New membrane | 27 | 28 | 15 | 4 | 6 |
| Sample 8 | Serum | 37 | 26 | 16 | 4 | 30 |
| | Standard membrane | 41 | 18 | 26 | 10 | 29 |
| | New membrane | 36 | 23 | 17 | 11 | 26 |
| Sample 9 | Serum | 7 | 4 | 9 | 2 | 9 |
| | Standard membrane | 11 | 7 | 18 | 6 | 18 |
| | New membrane | 9 | 5 | 13 | 4 | 13 |
| Sample 10 | Serum | 7 | 5 | 7 | 3 | 12 |
| | Standard membrane | 14 | 12 | 22 | 8 | 21 |
| | New membrane | 11 | 10 | 14 | 4 | 13 |
| Sample 11 | Serum | 3 | 3 | 5 | 2 | 12 |
| | Standard membrane | 13 | 11 | 28 | 11 | 23 |
| | New membrane | 8 | 7 | 14 | 6 | 14 |
| Sample 12 | Serum | 40 | 18 | 7 | 17 | 38 |
| | Standard membrane | 32 | 17 | 23 | 14 | 34 |
| | New- membrane | 35 | 16 | 10 | 13 | 36 |
| Sample 13 | Serum | 18 | 6 | 6 | 5 | 10 |
| | Standard membrane | 20 | 11 | 17 | 17 | 21 |
| | New membrane | 18 | 8 | 11 | 9 | 13 |
| Sample 14 | Serum | 23 | 28 | 36 | 5 | 8 |
| | Standard membrane | 22 | 24 | 34 | 10 | 18 |
| | New membrane | 20 | 23 | 30 | 6 | 10 |
| Sample 15 | Serum | 13 | 16 | 7 | 3 | 15 |
| | Standard membrane | 16 | 14 | 23 | 8 | 21 |
| | New membrane | 13 | 13 | 13 | 7 | 16 |
| Sample 16 | Serum | 6 | 4 | 5 | 6 | 9 |
| | Standard membrane | 12 | 8 | 21 | 9 | 18 |
| | New membrane | 8 | 5 | 9 | 8 | 11 |
| Sample 17 | Serum | 48 | 5 | 7 | 6 | 9 |
| | Standard membrane | 37 | 10 | 21 | 10 | 18 |
| | New membrane | 42 | 6 | 13 | 7 | 13 |
| Sample 18 | Serum | 26 | 7 | 11 | 5 | 18 |
| | Standard membrane | 21 | 11 | 24 | 8 | 23 |
| | New membrane | 21 | 9 | 17 | 7 | 17 |
| Sample 19 | Serum | 6 | 2 | 5 | 4 | 32 |
| | Standard membrane | 10 | 7 | 19 | 7 | 31 |
| | New membrane | 8 | 5 | 10 | 5 | 29 |
| Sample 20 | Serum | 14 | 6 | 7 | 4 | 16 |
| | Standard membrane | 13 | 8 | 13 | 11 | 19 |
| | New membrane | 11 | 7 | 10 | 7 | 15 |
| Sample 21 | Serum | 8 | 10 | 7 | 4 | 5 |
| | Standard membrane | 13 | 13 | 22 | 9 | 17 |
| | New membrane | 10 | 10 | 9 | 6 | 10 |
| | | | | | Guinea | |
| | | Walnut | Watermelon | Kiwi fruit | fowl | Rabbit |
| Sample 1 | Serum | 6 | 5 | 9 | 21 | 8 |
| | Standard membrane | 9 | 7 | 13 | 29 | 12 |
| | New membrane | 5 | 4 | 8 | 22 | 7 |
| Sample 2 | Serum | 11 | 4 | 4 | 7 | 2 |
| | Standard membrane | 13 | 6 | 11 | 22 | 10 |
| | New membrane | 12 | 3 | 7 | 12 | 4 |
| Sample 3 | Serum | 0 | 7 | 23 | 9 | 4 |
| | Standard membrane | 6 | 11 | 21 | 21 | 9 |
| | New membrane | 4 | 3 | 20 | 12 | 5 |
| Sample 4 | Serum | 13 | 4 | 31 | 13 | 3 |
| | Standard membrane | 11 | 7 | 28 | 26 | 10 |
| | New membrane | 8 | 5 | 28 | 18 | 7 |
| Sample 5 | Serum | 2 | 2 | 3 | 6 | 6 |
| | Standard membrane | 5 | 5 | 9 | 22 | 12 |
| | New membrane | 5 | 3 | 5 | 12 | 8 |
| Sample 6 | Serum | 5 | 5 | 22 | 6 | 3 |
| | Standard membrane | 8 | 11 | 20 | 22 | 9 |
| | New membrane | 3 | 6 | 18 | 10 | 4 |
| Sample 7 | Serum | 28 | 6 | 17 | 15 | 5 |
| | Standard membrane | 16 | 7 | 18 | 27 | 11 |
| | New membrane | 15 | 5 | 16 | 18 | 6 |
| Sample 8 | Serum | 15 | 14 | 32 | 7 | 4 |
| | Standard membrane | 15 | 14 | 27 | 18 | 8 |
| | New membrane | 15 | 13 | 27 | 10 | 4 |
| Sample 9 | Serum | 7 | 3 | 4 | 6 | 2 |
| | Standard membrane | 7 | 7 | 12 | 15 | 6 |
| | New membrane | 3 | 5 | 8 | 9 | 3 |
| Sample 10 | Serum | 5 | 8 | 6 | 7 | 5 |
| | Standard membrane | 11 | 11 | 13 | 18 | 9 |
| | New membrane | 8 | 8 | 8 | 10 | 7 |
| Sample 11 | Serum | 5 | 4 | 13 | 6 | 4 |
| | Standard membrane | 13 | 9 | 18 | 18 | 8 |
| | New membrane | 9 | 5 | 13 | 10 | 5 |
| Sample 12 | Serum | 33 | 12 | 17 | 3 | 3 |
| | Standard membrane | 30 | 13 | 19 | 12 | 5 |
| | New membrane | 33 | 13 | 17 | 6 | 3 |
| Sample 13 | Serum | 7 | 12 | 13 | 6 | 4 |
| | Standard membrane | 13 | 13 | 18 | 21 | 10 |
| | New membrane | 11 | 11 | 13 | 10 | 4 |
| Sample 14 | Serum | 15 | 10 | 8 | 27 | 20 |
| | Standard membrane | 15 | 11 | 14 | 26 | 15 |
| | New membrane | 13 | 7 | 8 | 23 | 16 |
| Sample 15 | Serum | 3 | 5 | 6 | 4 | 2 |
| | Standard membrane | 8 | 8 | 14 | 15 | 7 |
| | New membrane | 6 | 6 | 10 | 8 | 3 |
| Sample 16 | Serum | 4 | 4 | 6 | 5 | 2 |
| | Standard membrane | 9 | 8 | 13 | 15 | 6 |
| | New membrane | 4 | 5 | 8 | 8 | 4 |
| Sample 17 | Serum | 5 | 3 | 22 | 5 | 3 |
| | Standard membrane | 9 | 8 | 22 | 16 | 6 |
| | New membrane | 9 | 4 | 20 | 9 | 5 |
| Sample 18 | Serum | 7 | 6 | 21 | 10 | 3 |
| | Standard membrane | 12 | 10 | 23 | 19 | 7 |
| | New membrane | 9 | 7 | 20 | 12 | 5 |
| Sample 19 | Serum | 10 | 3 | 7 | 5 | 1 |
| | Standard membrane | 13 | 5 | 15 | 16 | 6 |
| | New membrane | 11 | 5 | 10 | 9 | 3 |
| Sample 20 | Serum | 4 | 10 | 15 | 8 | 4 |
| | Standard membrane | 8 | 10 | 18 | 16 | 7 |
| | New membrane | 7 | 8 | 14 | 9 | 5 |
| Sample 21 | Serum | 4 | 4 | 5 | 11 | 5 |
| | Standard membrane | 8 | 10 | 15 | 21 | 10 |
| | New membrane | 6 | 5 | 9 | 13 | 5 |

| | | Crab | Rose fish | Chives | Papaya |
|---|---|---|---|---|---|
| Sample 1 | Serum | 3 | 10 | 6 | 3 |
| | Standard membrane | 8 | 13 | 10 | 10 |
| | New membrane | 6 | 9 | 7 | 7 |
| Sample 2 | Serum | 9 | 4 | 3 | 3 |
| | Standard membrane | 11 | 10 | 10 | 11 |
| | New membrane | 8 | 6 | 5 | 7 |
| Sample 3 | Serum | 9 | 6 | 7 | 4 |
| | Standard membrane | 9 | 11 | 11 | 11 |
| | New membrane | 7 | 7 | 9 | 9 |
| Sample 4 | Serum | 1 | 3 | 5 | 4 |
| | Standard membrane | 6 | 9 | 10 | 10 |
| | New membrane | 2 | 6 | 5 | 6 |
| Sample 5 | Serum | 3 | 4 | 4 | 2 |
| | Standard membrane | 7 | 9 | 9 | 9 |
| | New membrane | 3 | 5 | 5 | 5 |
| Sample 6 | Serum | 1 | 4 | 4 | 3 |
| | Standard membrane | 5 | 9 | 10 | 9 |
| | New membrane | 3 | 5 | 5 | 5 |
| Sample 7 | Serum | 3 | 8 | 5 | 8 |
| | Standard membrane | 9 | 12 | 13 | 16 |
| | New membrane | 5 | 7 | 7 | 12 |
| Sample 8 | Serum | 7 | 29 | 10 | 11 |
| | Standard membrane | 9 | 26 | 12 | 15 |
| | New membrane | 8 | 24 | 10 | 15 |
| Sample 9 | Serum | 2 | 5 | 2 | 4 |
| | Standard membrane | 6 | 9 | 8 | 10 |
| | New membrane | 2 | 5 | 5 | 5 |
| Sample 10 | Serum | 8 | 9 | 7 | 3 |
| | Standard membrane | 10 | 11 | 7 | 9 |
| | New membrane | 8 | 7 | 7 | 8 |
| Sample 11 | Serum | 2 | 5 | 3 | 4 |
| | Standard membrane | 7 | 9 | 10 | 10 |
| | New membrane | 4 | 6 | 6 | 7 |
| Sample 12 | Serum | 4 | 8 | 10 | 6 |
| | Standard membrane | 7 | 12 | 13 | 12 |
| | New membrane | 4 | 8 | 13 | 11 |
| Sample 13 | Serum | 5 | 8 | 4 | 3 |
| | Standard membrane | 9 | 13 | 12 | 12 |
| | New membrane | 4 | 9 | 6 | 6 |
| Sample 14 | Serum | 3 | 5 | 7 | 10 |
| | Standard membrane | 8 | 10 | 10 | 14 |
| | New membrane | 5 | 6 | 9 | 13 |
| Sample 15 | Serum | 1 | 3 | 4 | 2 |
| | Standard membrane | 5 | 6 | 5 | 9 |
| | New membrane | 3 | 4 | 5 | 6 |
| Sample 16 | Serum | 1 | 2 | 3 | 4 |
| | Standard membrane | 5 | 6 | 6 | 8 |
| | New membrane | 2 | 4 | 4 | 4 |
| Sample 17 | Serum | 0 | 17 | 6 | 8 |
| | Standard membrane | 5 | 11 | 8 | 11 |
| | New membrane | 2 | 12 | 7 | 9 |
| Sample 18 | Serum | 4 | 4 | 4 | 5 |
| | Standard membrane | 8 | 9 | 9 | 13 |
| | New membrane | 5 | 6 | 7 | 8 |
| Sample 19 | Serum | 2 | 3 | 3 | 1 |
| | Standard membrane | 5 | 7 | 7 | 7 |
| | New membrane | 4 | 5 | 3 | 5 |
| Sample 20 | Serum | 3 | 7 | 3 | 2 |
| | Standard membrane | 7 | 8 | 5 | 5 |
| | New membrane | 4 | 4 | 5 | 5 |
| Sample 21 | Serum | 8 | 4 | 3 | 2 |
| | Standard membrane | 9 | 8 | 8 | 10 |
| | New membrane | 8 | 5 | 5 | 5 |

| | | Chicken | Cow | Total intensity over all |
|---|---|---|---|---|
| | | protein | milk | parameters per sample |
| Sample 1 | Serum | 75 | 129 | 352 |
| | Standard membrane | 67 | 125 | 389 |
| | New membrane | 69 | 126 | 345 |
| Sample 2 | Serum | 67 | 52 | 211 |
| | Standard membrane | 57 | 42 | 281 |
| | New membrane | 63 | 50 | 226 |
| Sample 3 | Serum | 65 | 15 | 218 |
| | Standard membrane | 51 | 7 | 252 |
| | New membrane | 57 | 11 | 214 |
| Sample 4 | Serum | 59 | 58 | 250 |
| | Standard membrane | 49 | 41 | 290 |
| | New membrane | 60 | 52 | 266 |
| Sample 5 | Serum | 3 | 26 | 88 |
| | Standard membrane | 8 | 14 | 172 |
| | New membrane | 5 | 19 | 113 |
| Sample 6 | Serum | 117 | 25 | 225 |
| | Standard membrane | 108 | 13 | 282 |
| | New membrane | 114 | 20 | 236 |
| Sample 7 | Serum | 69 | 80 | 319 |
| | Standard membrane | 62 | 60 | 343 |
| | New membrane | 70 | 76 | 317 |
| Sample 8 | Serum | 105 | 80 | 427 |
| | Standard membrane | 94 | 52 | 414 |
| | New membrane | 97 | 65 | 401 |
| Sample 9 | Serum | 31 | 6 | 98 |
| | Standard membrane | 21 | 2 | 163 |
| | New membrane | 27 | 4 | 120 |
| Sample 10 | Serum | 56 | 22 | 166 |
| | Standard membrane | 63 | 17 | 256 |
| | New membrane | 71 | 22 | 216 |
| Sample 11 | Serum | 60 | 40 | 171 |
| | Standard membrane | 68 | 37 | 293 |
| | New membrane | 73 | 44 | 231 |
| Sample 12 | Serum | 84 | 40 | 335 |
| | Standard membrane | 68 | 23 | 334 |
| | New membrane | 81 | 36 | 335 |
| Sample 13 | Serum | 18 | 58 | 183 |
| | Standard membrane | 18 | 40 | 260 |
| | New membrane | 18 | 50 | 201 |
| Sample 14 | Serum | 78 | 19 | 302 |
| | Standard membrane | 63 | 10 | 304 |
| | New membrane | 65 | 12 | 266 |
| Sample 15 | Serum | 77 | 21 | 182 |
| | Standard membrane | 58 | 12 | 229 |
| | New membrane | 62 | 15 | 190 |
| Sample 16 | Serum | 59 | 9 | 129 |
| | Standard membrane | 47 | 5 | 196 |
| | New membrane | 49 | 7 | 140 |
| Sample 17 | Serum | 81 | 35 | 260 |
| | Standard membrane | 72 | 20 | 284 |
| | New membrane | 79 | 28 | 265 |
| Sample 18 | Serum | 56 | 98 | 285 |
| | Standard membrane | 47 | 83 | 327 |
| | New membrane | 54 | 88 | 292 |
| Sample 19 | Serum | 83 | 7 | 174 |
| | Standard membrane | 74 | 4 | 233 |
| | New membrane | 74 | 5 | 191 |
| Sample 20 | Serum | 90 | 30 | 223 |
| | Standard membrane | 69 | 14 | 231 |
| | New membrane | 78 | 17 | 206 |
| Sample 21 | Serum | 73 | 17 | 170 |
| | Standard membrane | 57 | 8 | 238 |
| | New membrane | 70 | 14 | 190 |

| | Total intensity over all | Deviation [%] of total intensity |
|---|---|---|
| | parameters per sample material | in comparison with serum |
| Serum | 4768 | |
| Standard membrane | 5771 | 17.38 |
| New membrane | 4961 | 3.89 |

In the comparison of the two different materials used to obtain the DBS, what became apparent in the case of the Ahlstrom 226 membrane, which is a filter paper based on 100% cotton, was a distinctly increased background signal in the majority of parameters regardless of the sample, which signal led to false-positive results. Only in the case of the two parameters chicken protein and cow milk did this problem not occur.

Averaged over all samples, the values obtained with the “standard membrane” deviate by more than 15% from the values obtained using serum samples.

Similar results were obtained using the Whatman 903 membrane, a further membrane for DBS applications, which is likewise based on cotton. The data therefor are not shown here.

It was possible to achieve distinctly better results using the “new membrane” (Porex BNW441435), which is based on non-woven polypropylene fibres. The values obtained using said membrane deviate, on average, only by less than 4 percent from the values obtained using serum.

1. A carrier comprising
at least one take-up region based on a non-woven polyolefin, the at least one take-up region containing dried-up capillary blood.

2. A drawing set, comprising:
a carrier comprising at least one take-up region based on a non-woven polyolefin,
a sterile pricking aid for drawing capillary blood, and
optionally a pipette.

3. A method, comprising:
applying a blood sample to a carrier comprising at least one take-up region based on a non-woven polyolefin or to the at least one take-up region; and
semi-quantitatively or quantitatively determining a concentration of an analyte in the blood sample.

4. A method for detecting an analyte in a blood sample, comprising:
a) providing a carrier comprising at least one take-up region based on anon-woven polyolefin,
b) applying a blood sample to the at least one take-up region,
c) drying the carrier to obtain a dried-up blood sample,
d) contacting the carrier or the at least one take-up region thereof comprising the dried-up blood sample from c) with a liquid which is suitable for taking up an analyte from the dried-up blood sample, and
e) detecting the analyte in the liquid.

5. The carrier according to claim 1, wherein the at least one take-up region is surrounded by a material having an absorptivity lower than an absorptivity of the at least one take-up region.

6. The carrier according to claim 1, comprising:
at least two take-up regions based on the non-woven polyolefin that are separated from one another.

7. The method according to claim 3, wherein the blood sample is capillary blood.

8. The method according to claim 7, wherein the capillary blood is untreated capillary blood.

9. The Carrier according to claim 1, wherein the non-woven polyolefin is polypropylene.

10. The method according to claim 4, wherein the detecting is a semi-quantitative detection.

11. The method according to claim 3, wherein the method is for a diagnosis of a food allergy or food intolerance.

12. The method according to claim 4, wherein the detecting is carried out with a diagnostically useful carrier configured to specifically capture an antibody.

13. The method according to claim 12, wherein the diagnostically useful carrier is selected from the group consisting of a blot, a bead, a microtiter plate, a test strip, a microarray, a glass slide, a biochip, and a lateral flow device.

14. The method according to claim 4, wherein the analyte is an antibody.

15. The method according to claim 14, wherein the antibody is an antibody against an allergen-containing and/or a protein-containing material based on a foodstuff.

16. The carrier according to claim 6, wherein the at least two take-up regions are separated from one another by a material having an absorptivity lower than absorptivities of the at least two take-up regions.

17. The method according to claim 13, wherein the diagnostically useful carrier is the blot selected from the group consisting of a line blot, a dot blot, a western blot, and a combination thereof.

18. The method according to claim 14, wherein the antibody is an IgG class antibody.

19. The method according to claim 15, wherein the foodstuff is selected from the group consisting of courgette, pepper, chicken meat, codfish, salmon, walnut, watermelon, kiwi fruit, guinea fowl, rabbit, crab, rose fish, chives, papaya, and a combination thereof.
